(19) United States
(12) Patent Application Publication
(10) Pub. No.: US 2026/0232348 A1
(43) Pub. Date: Aug. 13, 2026

(54) DYNAMIC OBTURATOR SYSTEM

(71) Applicant: LSI Solutions, Inc., Victor, NY (US)

(72) Inventor: Jude S. Sauer, Pittsford, NY (US)

(21) Appl. No.: 19/440,310

(22) Filed: Jan. 5, 2026

Related U.S. Application Data
(60) Provisional application No. 63/741,591, filed on Jan. 3, 2025.

(51) Int. Cl. A61B 17/34

(52) U.S. Cl. CPC A61B 17/3423; A61B 2017/3454

(57) ABSTRACT

An insertion system includes an obturator assembly includes an elongated inner obturator member that is inserted into an explainable obturator member, and the obturator assembly is inserted into a cannula member to comprise an insertion system. During a procedure, the inner obturator member is advanced distally relative to the expandable obturator member such that a distal end of a body portion of the inner obturator member contacts contact portions of tip portions of arm members 34 of the expandable obturator member, thereby causing the expandable obturator member to radially expand to a gauge larger than the outer diameter of the cannula member 12, thus creating a dynamic leading edge that is retracted automatically upon removal of the inner obturator member.

### RELATED APPLICATIONS

This application claims priority to U.S. Provisional Patent Application No. 63/741,591, filed Jan. 3, 2026, which is hereby incorporated by reference in its entirety.

### FIELD

The claimed invention relates to surgical devices, and more specifically to a surgical access system.

### BACKGROUND

A laparoscopic cannula is a hollow, tube-like port (often plastic or metal) used in minimally invasive surgery to create a pathway into the body cavity, allowing surgeons to insert instruments during a procedure. Typically, the cannula is specifically sized to accommodate different tools and procedures, and works with a sharp trocar to pierce tissue initially.

Conventional cannulas present several disadvantages that can impact patient safety and procedural efficacy. One significant issue is the potential for trauma during insertion and manipulation. The design of these cannulas often includes corners where the cannula meets the obturator, which can cause tissue damage upon entry or movement within the body. This trauma can lead to increased bleeding, prolonged recovery times, and heightened patient discomfort.

Additionally, the edges can contribute to inadvertent perforation of surrounding structures, increasing the likelihood of adverse events such as infection or internal bleeding. In procedures requiring precision, any trauma inflicted by the cannula can compromise the surgical outcome and necessitate additional interventions. Therefore, the need for improved cannula designs that minimize sharp transitions is crucial in advancing surgical techniques and reducing patient trauma.

### BRIEF DESCRIPTION OF THE DRAWINGS

FIGS. 1A to 1C, 1E to 1F are various views of an embodiment of an obturator assembly in a first position;

FIG. 1D is a cross-sectional view of the embodiment of the obturator assembly in the first position taken along section line 1D-1D of FIG. 1A;

FIGS. 2A to 2D and 2G are various views of an embodiment of an explainable obturator member of the obturator assembly of FIG. 1A;

FIGS. 2E and 2H are cross-sectional view of the embodiment of the explainable obturator member taken along section line 2E-2E of FIG. 2A;

FIG. 2F is a detail of the cross-sectional view of FIG. 2E;

FIGS. 3A to 3C are various views of an embodiment of a cannula member of the insertion system of FIGS. 6A, 6B, 7A, 8A, and 8B;

FIG. 3D is a cross-sectional view of the embodiment of the cannula member taken along section line 3D-3D of FIG. 3A; and

FIGS. 4A to 4E are various views of an embodiment of an inner obturator member of the obturator assembly of FIG. 1A.

FIG. 5 is a cross-sectional view of the embodiment of the insertion system of FIGS. 6A and 8A taken along section line 1D-1D of the obturator assembly FIG. 1A in the first position;

FIGS. 6A and 8A are views of an embodiment of an insertion system that includes the obturator assembly of FIG. 1A in a first position;

FIGS. 6B and 8B are views of an embodiment of the insertion system that includes the obturator assembly of FIG. 1A in a second position; and

FIG. 7A is a side view of the embodiment of the obturator assembly of FIG. 1A in a second position; and

FIG. 7B is a cross-sectional view of the embodiment of the obturator assembly of FIG. 1A taken along section line 1D-1D in the second position.

### DETAILED DESCRIPTION

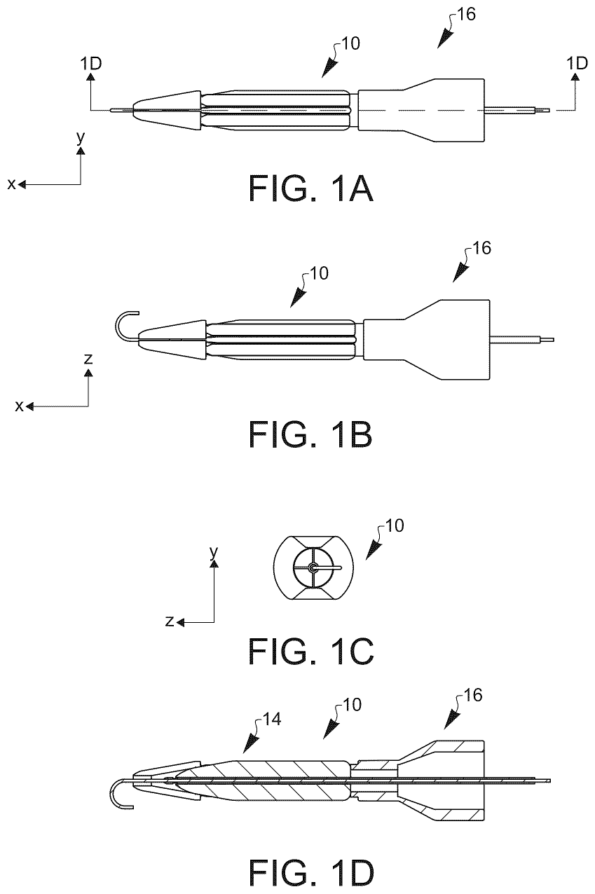

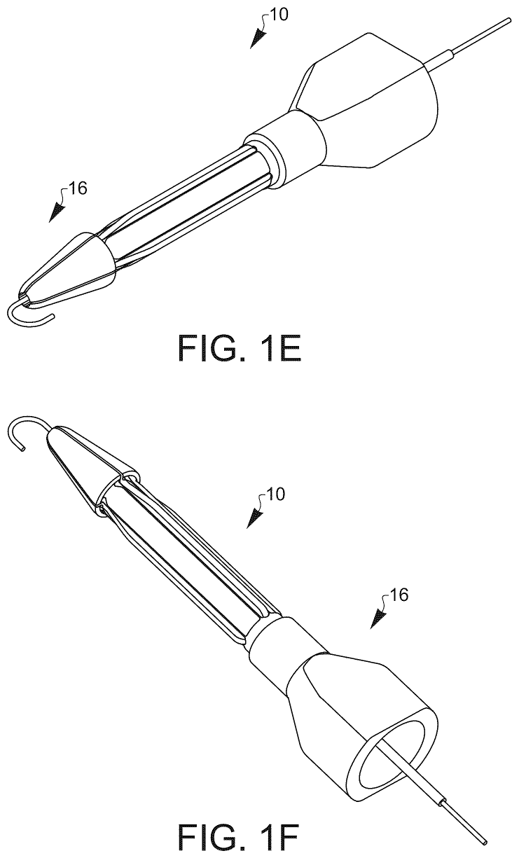

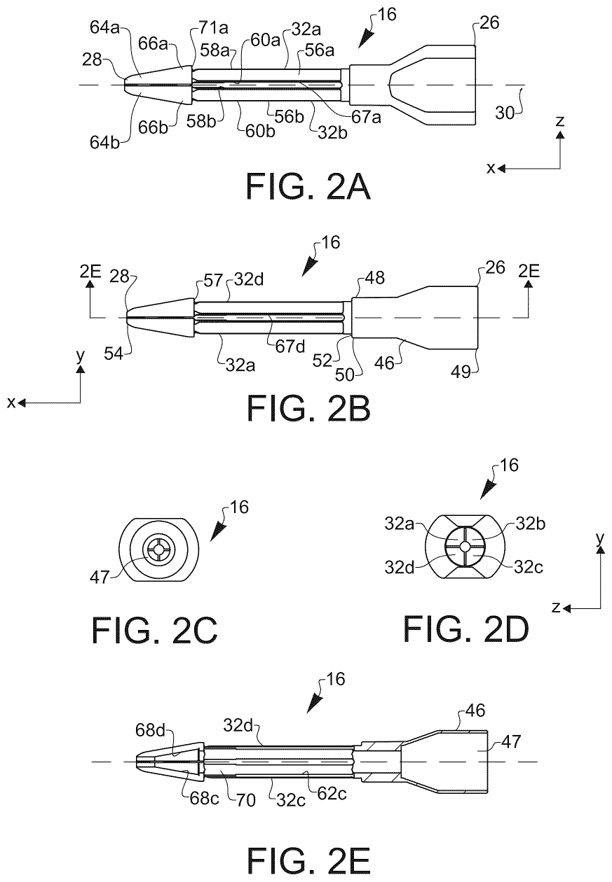

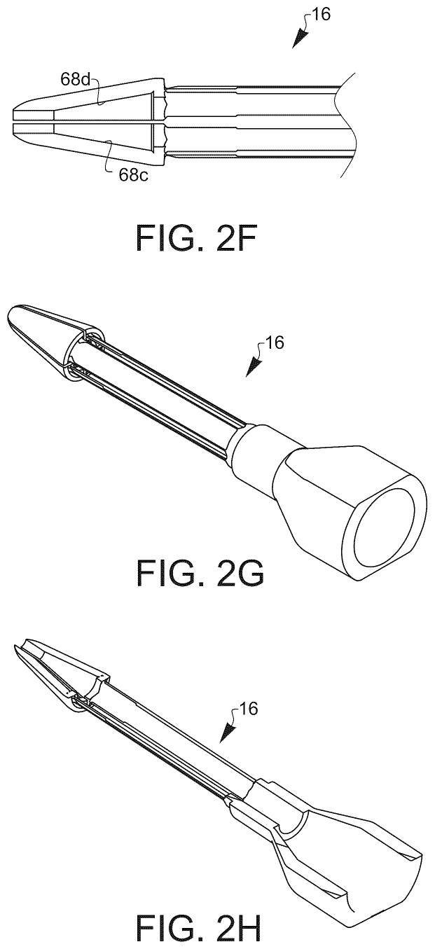

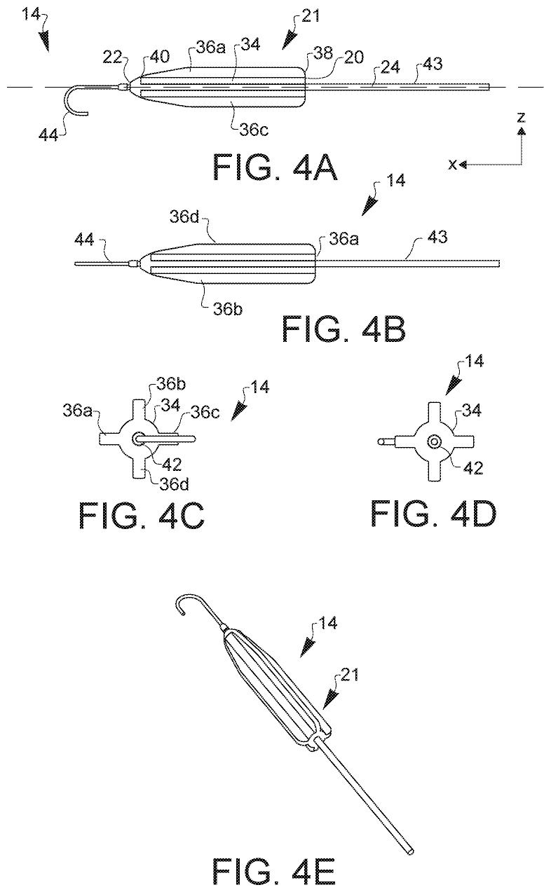

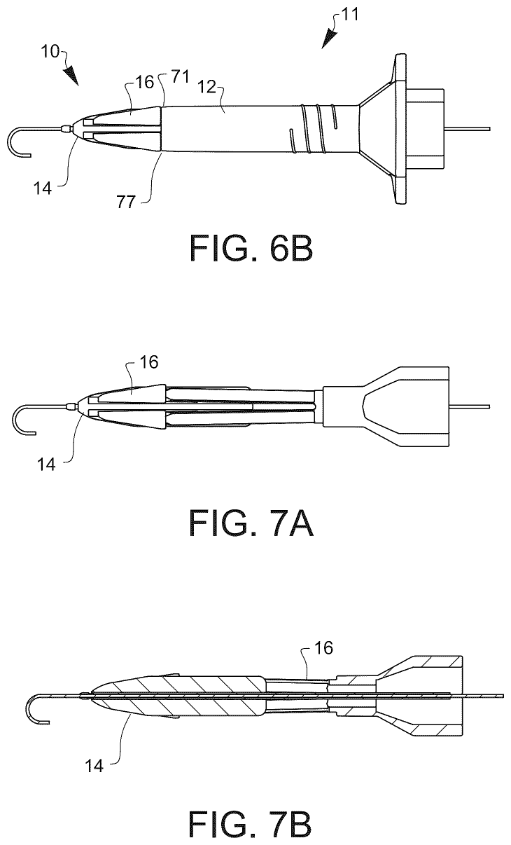

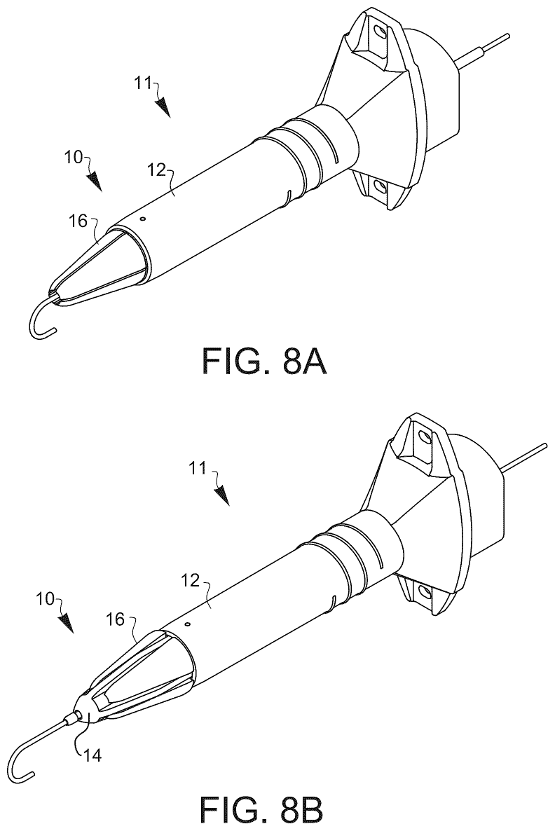

FIGS. 1A to 1C, and 1E to 1F illustrate various views of an embodiment of an obturator assembly 10 in a first position. The obturator assembly 10 includes an inner obturator member 14 that is inserted into an explainable obturator member 16, and the obturator assembly 10 may be inserted into a cannula member 12 to comprise an insertion system 11, illustrated in the first position in FIGS. 6A and 8A. As illustrated in FIG. 4A, a distal end 22 of a body portion 21 of the inner obturator member 14 may be advanced distally within the explainable obturator member 16 (illustrated in FIG. 2A) such that the distal end 22 of the body portion 21 of inner obturator member 14 contacts portions of two or more arm members 32 disposed at a distal end 28 of the expandable obturator member 16, thereby expanding the portions of two or more arm members 32 as the inner obturator member 14 is advanced distally until the obturator assembly 10 is in a second position illustrated in FIG. 7A. With the obturator assembly 10 inserted into the cannula member 12 to comprise an insertion system 11, the insertion system 11 provides an expanding obturator tip for idealized atraumatic insertion through the chest wall. In particular, the advancing distal end 22 of the body portion 21 of the inner obturator member 14 causes the expandable obturator member 16 to open radially to a gauge slightly larger than the outer diameter of the cannula member 12, thus creating a dynamic and contoured leading edge that is retracted automatically upon removal of the inner obturator member 14, as will be described in more detail in following sections.

Turning to the obturator assembly 10 in more detail, and with reference to FIGS. 4A to 4E, the inner obturator member 14 includes a body portion 21 that extends from a proximal end 20 to the distal end 22 along an inner obturator axis 24 that may be parallel to the X-axis of the reference coordinate systems of FIGS. 4A and 1A. The body portion 21 includes a center portion 34 that extends from the proximal end 20 to the distal end 22 along the inner obturator axis 24, and the center portion 21 may be cylindrical. An end portion of the body portion 21 at the distal end 22 is chamfered, radiused, or gradually reduced in diameter so as to reduce trauma when introduced into a patient. Two or more elongated fin members 36 (e.g., four, six or eight fin members 36) may extend along a corresponding portion of an external surface of the body portion 21, and in some embodiments, the inner obturator member 14 may have four fin members 36a, 36b, 36c, 36d that may be identical and that may be radially-arrayed at even intervals (e.g., 90° intervals) when viewed along the X-axis. Each of the fin members 36a, 36b, 36c, 36d may extend from a proximal end 38 to the distal end 40 along the inner obturator axis 24, and the proximal end 38 of the fin members 36a, 36b, 36c, 36d may be at or adjacent to the proximal end 20 of the body portion 21. The lateral height of each of the fin members 36a, 36b, 36c, 36d may be between 50% and 100% of a diameter of the center portion 34. A distal portion of each of the fin members 36a, 36b, 36c, 36d may be tapered and increase in height from the distal end 40 towards the proximal end 38 so as to reduce trauma when introduced into a patient. The fin members 36a, 36b, 36c, 36d may also have the same width (i.e., the dimension normal to the inner obturator axis 24). The fin members 36a, 36b, 36c, 36d and the center portion 34 may be integrally formed to comprise the body portion 21.

As illustrated in FIGS. 4C and 4D, the inner obturator member 14 may also include a center aperture 42 may extend through the center portion 34 from the proximal end 20 to the distal end 22 of the body portion 21 along the inner obturator axis 24, and a tube 43 may be inserted into, and may extend within all or part of, the center aperture 42. The tube 43 may receive a guide wire 44 for advancing the obturator assembly 10 (or any of the inner obturator member 14 or the explainable obturator member 16) to a desired treatment area in a known manner.

With reference to FIGS. 2A to 2D and 2G, the obturator assembly 10 also includes the expandable obturator member 16 that extends from a proximal end 26 to the distal end 28 along an expandable obturator axis 30 that may be parallel to the X-axis of the reference coordinate systems of FIGS. 4A and 1A, and the expandable obturator axis 30 may be coaxially aligned with the inner obturator axis 24 during insertion and use. The expandable obturator member 16 may include a base portion 46 that extends from a distal end 50 (at an intermediate point 48 of the obturator member 16) to a proximal end 49 at the proximal end 26 of the expandable obturator member 16. A base aperture 47 (see FIG. 2C) may extend through the base portion 46 from the proximal end 49 to the distal end 50, and the base aperture 47 may be defined by one or more surfaces of the wall(s) that cooperate to form the base portion 46.

With reference to FIG. 2B, the expandable obturator member 16 may also include two or more elongated arm members 32 (e.g., four, six or eight arm members 32) that each extends from a proximal end 52 to the distal end 54 parallel to the expandable obturator axis 30, and the proximal end 52 may be at or adjacent to the intermediate point 48 and/or the distal end 50 of the base portion 46. In some embodiments, the expandable obturator member 16 may have four arm members 32a, 32b, 32c, 32d that may be identical and that may be radially-arrayed at even intervals (e.g., 90° intervals) when viewed along the X-axis.

Each of the arm members 32 may include a stem portion 56 that extends from a proximal end to distal end. The proximal end may be at the proximal end 52 of the arm member 32 and the distal end may be at a second intermediate point 57 between the proximal end 52 and the distal end 54. Each of the stem portions 56 may include opposing first and second lateral edges 58, 60. For example, the first stem portion 56a may be partially defined by opposing first and second lateral edges 58a, 60a, and the second stem portion 56b may be partially defined by opposing first and second lateral edges 58b, 60b, and the second lateral edge 60a of the first stem portion 56a may be adjacent to the first lateral edge 58b of the second stem portion 56b such that a space exists between the first lateral edge 58b of the second stem portion 56b and the second lateral edge 60a of the first stem portion 56a. This space forms a proximal portion of a gap 67a, 67b, 67c, 67d between each of the adjacent stem portions 65, and the proximal portion of the gap 67 may be equal and may have a width that is slightly greater than the width of the fin members 36a, 36b, 36c, 36d of the body portion 21 of the inner obturator member 14.

Each of the stem portions 56 is partially defined by an inner surface 62 that extends between the opposing first and second lateral edges 58, 60, and the inner surfaces 62a, 62b, etc. of each of the stem portions 56a, 56b, etc. cooperate to form a substantially circular cross-sectional shape when viewed normal to the X-axis of the reference coordinate system of FIG. 2A. In other embodiments, the inner surfaces 62a, 62b, etc. may cooperate to form an oval, diamond, or other cross-sectional shape when viewed normal to the X-axis of the reference coordinate system of FIG. 2A.

Each of the arm members 32 may also include a tip portion 64 that extends from a proximal end to distal end. The proximal end may be at the distal end of the stem portion 56 (and/or the second intermediate point 57) and the distal end may be at the distal end 54 of the arm member 32. The tip portions 64a, 64b, etc. may have outer surfaces 66 that cooperate to form a frusto-conical shape having a circular cross-sectional shape (when viewed along the X-axis of the reference coordinate system of FIG. 2A) with a diameter that increases from the distal end to the proximal end of each tip portion 64. A ridge, or lip 71a, 71b, etc., may be formed at the proximal end of each of the tip portions 64a, 64b, etc. The outer surfaces 66 may cooperate to form a circular (or substantially circular) cross-sectional shape when viewed along the expandable obturator axis 30 at the proximal ends of the tip portion 64 (i.e., the tip portions 64a, 64b, etc.)

The tip portions 64a, 64b, etc. may also each include an inner surface 68 that cooperate to form a contoured, gradually decreasing (from the proximal end to the distal end) conduit through the tip portion 64a. The inner surfaces 68a, 68b, etc. and the outer surfaces 66 may cooperate to form a central aperture 70 (see FIG. 2E) that may also include the base aperture 47, and the central aperture 70 may extend from the proximal end 26 to the distal end 28 of the expandable obturator member 16. Lateral surfaces defining the tip portions 64a, 64b, etc. may further define a distal portion of the gap 67, along with the proximal portion of the 67 between the stem portions 56.

So configured, the arm members 32 may act as leaf springs such that (a) when a radially-outward force (that is normal to the expandable obturator axis 30) is applied to any arm member 32 (on a portion of the arm member 32 that is distal to the proximal end 52), the distal end 54 of that arm member 32 displaces away from the expandable obturator axis 30) and/or (b) when a radially-inward force (that is normal to the expandable obturator axis 30) is applied to any arm member 32 (on a portion of the arm member 32 that is distal to the proximal end 52), the distal end 54 of that arm member 32 displaces towards the expandable obturator axis 30).

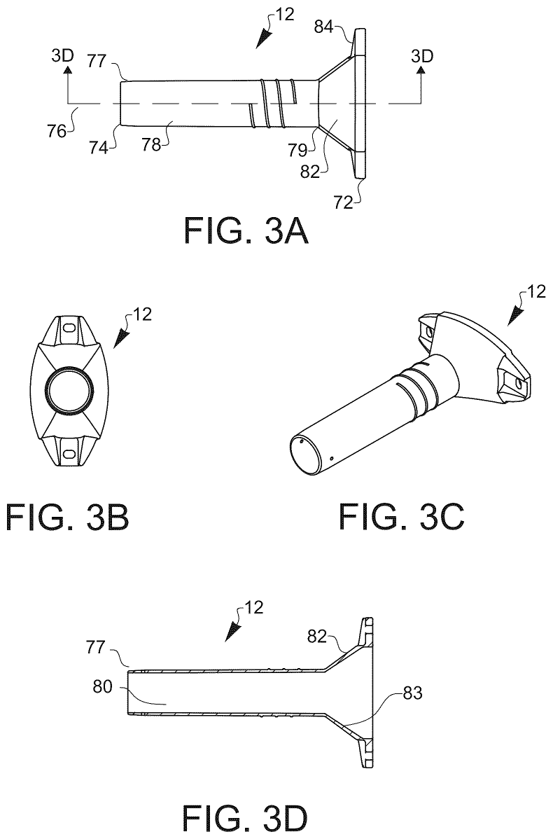

With reference to FIGS. 3A to 3D, the insertion system 11 also includes the cannula member 12 that extends from a proximal end 72 to a distal end 74 along a cannula axis 76, and the cannula axis 76 may be coaxially aligned with the expandable obturator axis 30 and/or with the inner obturator axis 24 during insertion and use. The cannula member 12 may include a body portion 78 that may extend from a proximal end to a distal end, and the distal end may be at the distal end 74 of the cannula member 12 and the proximal end may be at an intermediate point 79. The body portion 78 may be hollow and may have one or more internal surfaces that may partially define a first portion of a cannula aperture 80, and the one or more internal surfaces may define a shape having a circular-cross section. The cannula aperture 80 may have any suitable size, such as 12 mm. The body portion 78 may have a constant cross-sectional shape or a portion of the body portion 78 may have a constant cross-sectional shape. An outer surface 77 of the body portion 78 may have a circular cross-sectional shape such that the outer surface 77 have a cylindrical shape.

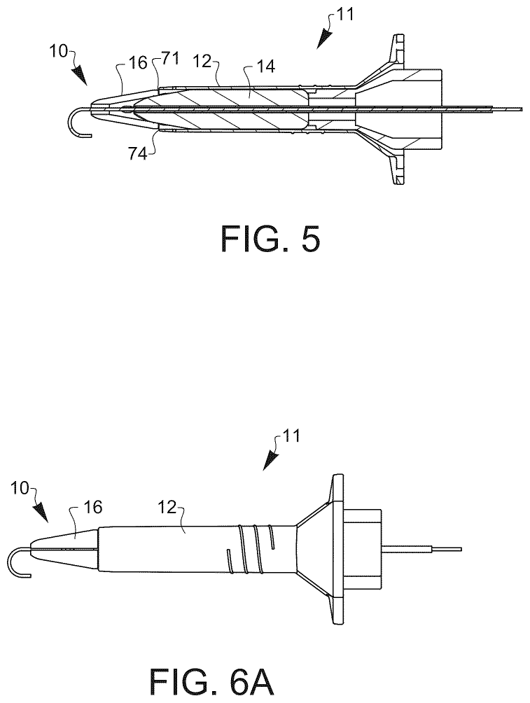

The cannula member 12 may also include an end portion 82 that may extend from a proximal end to a distal end, and the distal end may be at the proximal end of the body portion 78 (i.e., at the at an intermediate point 79) and the proximal end may be at the proximal end 72 of the cannula member 12. The end portion 82 may have one or more internal surfaces 83 that may define a second portion of the cannula aperture 80, and the one or more internal surfaces 83 of the end portion 82 may define a funnel-type shape that becomes wider as the end portion 82 extends proximally, and the one or more internal surfaces 83 of the end portion 82 are configured to guide the obturator assembly 10 into the cannula aperture 80. In some embodiments, the one or more internal surfaces of the end portion 82 may have the general shape of the external surfaces defining the a distal portion of the base portion 46 of the expandable obturator member 16 such that all or a portion of the external surfaces of the base portion 46 of the expandable obturator member 16 may be received into the second portion of the cannula aperture 80 and/or may contact all or a portion of the one or more internal surfaces of the end portion 82 when the obturator assembly 10 is inserted into the cannula member 12, as illustrated in the cross-sectional view of FIG. 5A. The cannula member 12 may also include a flange portion 84 at the proximal end 72 of the cannula member 12.

To assemble the obturator assembly 10, the distal end 22 of the body portion 21 of the inner obturator member 14 is inserted into the base aperture 47 of the base portion 46 of the expandable obturator member 16, and the distal end 22 of the body portion 21 of the inner obturator member 14 is advanced distally with each of the fin members 36a, 36b, 36c, 36d of the body portion 21 of the inner obturator member 14 aligned with a corresponding one of the gaps 67a, 67b, 67c, 67d between each of the adjacent stem portions 65 of the expandable obturator member 16. The distal end 22 of the body portion 21 of the inner obturator member 14 is advanced distally until each of the fin members 36a, 36b, 36c, 36d of the body portion 21 of the inner obturator member 14 is received into at least a portion of the corresponding one of the gaps 67a, 67b, 67c, 67d of the expandable obturator member 16. In particular, at least a portion of the distal portion of each of the fin members 36a, 36b, 36c, 36d may be received in the corresponding one of the gaps 67a, 67b, 67c, 67d, and the proximal end 38 of the fin members 36a, 36b, 36c, 36d may be at or distally offset from the proximal end of the stem portions 56 that define the gaps 67. In this first position, a portion of the distal end 22 of the body portion 21 of the inner obturator member 14 is in contact with (or is slightly proximally offset from) a corresponding portion of each of the inner surfaces 68 (see FIG. 1D) of the tip portions 64 of the arm members 34 of the expandable obturator member 16.

During a minimally-invasive procedure, the obturator assembly 10 may be assembled when disposed within the cannula member 12, and the obturator assembly 10 may be displaced from the first position of FIG. 1A to the second position of FIG. 7A. For example, the expandable obturator member 16 may be advanced distally into the cannula aperture 80 of the cannula member 12 until the lip 71 at the proximal end of each of the tip portions 64 extend to (or slightly distal to) the distal end 74 of the cannula member 12.

The inner obturator member 14 may then be advanced distally relative to the expandable obturator member 16, as described above. As the inner obturator member 14 is inserted into the expandable obturator member 16, the distal end 22 of the body portion 21 of the inner obturator member 14 may contact the corresponding portion of each of the inner surfaces 68 (see FIG. 2E) of the tip portions 64 of the arm members 34 of the expandable obturator member 16 to cause the distal ends of the arm member 32 to outwardly displace relative to the proximal ends, allowing for further distal displacement of the distal end 22 of the body portion 21 of the inner obturator member 14.

When the inner obturator member 14 is fully inserted into the expandable obturator member 16 (i.e., when the obturator assembly 10 is in the second position), portions at distal ends of the outer surfaces of each of the stem portions 56 of the arm members 32 may contact (or be adjacent to) corresponding portions of the internal surfaces that may partially define a portion of the cannula aperture 80 of the cannula member 12. With the inner obturator member 14 fully inserted into the expandable obturator member 16, the lip 71 at the proximal end of each of the tip portions 64 may be in contact with or immediately adjacent to the distal end of the body portion 78 at the distal end 74 of the cannula member 12.

So configured, the proximal ends of the outer surfaces 66 of the tip portions 64, which have a circular (or substantially circular) cross-sectional shape, may extend radially beyond the cylindrical outer surface 77 at the distal end of the body portion 78 of the cannula member 12, as illustrated in FIG. 6B. In this configuration of the insertion system 11 in which the obturator assembly 10 is in the second position, the advanced distal end 22 of the body portion 21 of the inner obturator member 14 creates a rounded or contoured leading edge of the insertion system 11 that minimizes trauma to tissue of the patient during insertion of the insertion system 11. Further, because the proximal ends of the outer surfaces 66 of the tip portions 64, extend radially beyond the cylindrical outer surface 77 at the distal end of the body portion 78 of the cannula member 12, there are no sharp edges that could case tissue damage during insertion or use of the insertion system 11.

To withdraw the obturator assembly 10 from the cannula member 12, the inner obturator member 14 may be pulled distally from the expandable obturator member 16. Without the radially outward force provided by contact with the inner obturator member 14, the lip 71 at the proximal end of each of the tip portions 64 may inwardly displace towards the expandable obturator axis 30, and the due to the leaf spring quality of the arm members 32 of the expandable obturator member 16, the expandable obturator member 16 may be proximally displaced within the cannula aperture 80 of the cannula member 12 until the expandable obturator member 16 is removed from the cannula member 12. So configured, the cannula member 12 may be sued to insert instruments, as is known in the art.

The insertion system 11 may be positioned adjacent to and advanced withing an incision of a patient in this configuration, or the cannula member 12 may be positioned within an incision in the patient first, and the obturator assembly 10 may be subsequently inserted.

The body portion 21 of the inner obturator member 14 may be formed or manufactured as a single, unitary part, and may be made from any suitable material, such as injection molded plastic or medical grade metal. Further, the expandable obturator member 16 14 may be formed or manufactured as a single, unitary part, and may be made from any suitable material, such as injection molded plastic or medical grade metal. In addition, the cannula member 12 may be formed or manufactured as a single, unitary part, and may be made from any suitable material, such as injection molded plastic or medical grade metal.

Various advantages of a surgical insertion system have been discussed above. Embodiments discussed herein have been described by way of example in this specification. It will be apparent to those skilled in the art that the foregoing detailed disclosure is intended to be presented by way of example only, and is not limiting. As just one example, although the end effectors in the discussed examples were often focused on the use of a scope, such systems could be used to position other types of surgical equipment. Various alterations, improvements, and modifications will occur and are intended to those skilled in the art, though not expressly stated herein. These alterations, improvements, and modifications are intended to be suggested hereby, and are within the spirit and the scope of the claimed invention. The drawings included herein are not necessarily drawn to scale. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. An insertion system for use in a surgical procedure, the insertion system 11 comprising:
an obturator assembly comprising:
an inner obturator member comprising:
a body portion extending from a proximal end to a distal end along an inner obturator axis, an end portion of the body portion at the distal end 22 is chamfered, radiused, or gradually reduced in diameter so as to reduce trauma when introduced into a patient, the body portion comprising:
a center portion extending from the proximal end to the distal end along the inner obturator axis;
four elongated fin members that each extends along a corresponding portion of an external surface of the body portion; and
a center aperture extending through the center portion from the proximal end to the distal end of the body portion along the inner obturator axis;
an expandable obturator member extending from a proximal end to a distal end along an expandable obturator axis, the explainable obturator member comprising:
a base portion that defines a base aperture;
four elongated arm members that each extends from a proximal end to a distal end along an axis, the proximal end being coupled to a distal end of the base portion, each of the arm members comprising:
a stem portion that extends from a proximal end to distal end, the proximal end disposed at the proximal end of the arm member,
a tip portion that extends from a proximal end to distal end, with the proximal end at the distal end of the stem portion and the distal end at the distal end of the arm member, the tip portions having an outer surface that forms a portion of a frusto-conical shape with a diameter that increases from the distal end to the proximal end of each tip portion, and wherein a lip is formed at the proximal end of tip portion,
wherein the inner obturator member is slidably disposed within the explainable obturator member such that each of the four elongated fin members are disposed within corresponding slots between adjacent arm members of the expandable obturator member, the inner obturator member being slidably disposed relative to the expandable obturator member from a first position of the obturator assembly, in which the axis of each of the arm members is parallel to the expandable obturator axis, to a second position of the obturator assembly, in which the distal end of each of the arm members is outwardly displaced relative to the first position; and
a cannula member extending from a proximal end to a distal end along a cannula axis, the cannula member including a body portion that extends from a proximal end to a distal end, the body portion defining at least a portion of a cannula aperture, and the distal end may of the body portion at the distal end of the cannula member,
wherein when a portion of the obturator assembly is disposed within the cannula aperture such that the distal end of the body portion of the cannula member is at or adjacent to the proximal end of each of the tip members of the arm members of the expandable obturator member such that when the obturator assembly is displaced from the first position to the second position, a proximal end of the frusto-conical shape formed by the outer surfaces of the tip members is disposed outward of a distal end of an outer surface of the body portion of the cannula member so as to reduce trauma to tissue of a patient when the insertion system is inserted into a body cavity of the patient.

2. The insertion system of claim 1, wherein the four fin members are radially-arrayed at even intervals when viewed along the inner obturator axis.

3. The insertion system of claim 1, wherein the expandable obturator axis is coaxially aligned with the inner obturator axis.

4. The insertion system of claim 1, wherein the base portion and the arm members of the expandable obturator member are formed as a single, unitary part.

5. The insertion system of claim 1, wherein the center portion and the fin members of the body portion of the inner obturator member are formed as a single, unitary part.

6. The insertion system of claim 1, wherein the outer surface of the body portion of the cannula member is cylindrical.

7. The insertion system of claim 1, the obturator assembly further comprising a guide wire having a portion extending through the center aperture of the body portion of the obturator member.

8. The insertion system of claim 1, wherein a proximal end of each of the fin members is at or adjacent to the proximal end of the body portion.

9. The insertion system of claim 8, wherein a distal portion of each of the fin members is tapered and increase in height from the distal end towards the proximal end so as to reduce trauma when introduced into a patient.

10. The insertion system of claim 1, wherein a lateral height of each of the fin members is between 50% and 100% of a diameter of the center portion.
